(19) United States
(12) Patent Application Publication
(10) Pub. No.: US 2023/0212508 A1
(43) Pub. Date: Jul. 6, 2023

(54) ADIPOSE TISSUE REGENERATION BASE MATERIAL

(71) Applicants: GUNZE LIMITED, Ayabe-shi, Kyoto (JP); KYOTO UNIVERSITY, Kyoto-shi, Kyoto (JP); KANSAI MEDICAL UNIVERSITY, Hirakata-shi, Osaka (JP)

(72) Inventors: Saki MATSUBARA, Kyoto (JP); Yuuki KATO, Kyoto (JP); Naoki MORIMOTO, Osaka (JP); Shuichi OGINO, Kyoto (JP)

(21) Appl. No.: 17/999,501

(22) PCT Filed: Feb. 26, 2021
(86) PCT No.: PCT/JP2021/007314

§ 371 Date: Nov. 21, 2022

(30) Foreign Application Priority Data
May 29, 2020 (JP) 2020-094690

(51) Int. Cl. C12N 5/00; C12M 1/00

(52) U.S. Cl. CPC C12N 5/0068; C12M 21/14; C12N 2533/40

(57) ABSTRACT

The present invention aims to provide an adipose tissue regeneration substrate that has high handleability and that enables regeneration of a large volume of adipose tissue in a normal shape. Provided is an adipose tissue regeneration substrate including: a plurality of granular bodies made of a bioabsorbable material, each granular body having an inner space and having on a surface a plurality of openings leading to the inner space; and a bag-shaped body made of a bioabsorbable material, the bag-shaped body having an opening and wrapping the granular bodies.

### TECHNICAL FIELD

The present invention relates to an adipose tissue regeneration substrate that has high handleability and that enables regeneration of a large volume of adipose tissue in a normal shape.

### BACKGROUND ART

Breast cancer is treated by surgical removal (surgical therapy) when cancer in the breast is difficult to cure by radiation or chemotherapy alone. In the past, a total mastectomy, which removes an entire breast including the lesion tissue, was common. In recent years, improved examination techniques have enabled earlier detection of small lesions, and enabled breast-conserving surgery, which removes only a tissue portion. However, breast-conserving surgery may still pose a psychological burden on the patient as it causes a depression in the resected area. To improve the quality of life (QOL), more and more patients are receiving breast reconstruction after surgical therapy.

Silicone implants are common in breast reconstruction. However, they are not bioabsorbable and remain in the body forever as foreign bodies, possibly causing postoperative leakage or infection due to foreign body reaction. There are also concerns for adverse effects such as allergic reactions and carcinogenesis caused by contact with such implants.

An alternative approach is to harvest adipose tissue from other sites and transplant it to the depressed portion. The transplanted tissue, however, may be quickly absorbed and form a depression again. Moreover, harvesting tissue creates a new wound, which can be undesirable from a quality-of-life standpoint.

To solve the issues with conventional breast reconstruction techniques, the present inventors have disclosed a member for breast reconstruction including a collagen-containing sponge inside a hollow polylactic acid granular body (Patent Literature 1). When the member for breast reconstruction of Patent Literature 1 is placed in a space formed by partial mastectomy, the surrounding cells enter the member for breast reconstruction and use the member as a scaffold for proliferation. This enables breast reconstruction without transplanting adipose tissue from other sites. Moreover, the member for breast reconstruction of Patent Literature 1 is highly safe because, being made of bioabsorbable materials, it is gradually absorbed into the body as the breast regeneration proceeds and eventually disappears.

### CITATION LIST

### Patent Literature

Patent Literature 1: JP 2016-140494 A

### SUMMARY OF INVENTION

### Technical Problem

The member for breast reconstruction of Patent Literature 1 is a very effective means for breast reconstruction as it can safely regenerate a breast from autologous, living cells. The member for breast reconstruction of Patent Literature 1, however, may have low ease of implanting because in large resection areas, the member for breast reconstruction needs to be placed in large amounts. In particular, breast cancer treatment combines cancer resection with radiation therapy so as to completely kill cancer cells after the resection. Since the radiation therapy hardens the skin, the skin is gradually expanded with a tissue expander or a similar device, and then incisions are made to remove the tissue expander and insert an implant. To actually place the small implant of Patent Literature 1 in large amounts with good cosmetic results, large incisions are required, which increases burden on the patient.

One solution for the above issue may be to make the member for breast reconstruction larger. However, too large a member for breast reconstruction may have not only low moldability but also low tissue regeneration performance because such a member makes it difficult for adipose tissue to regenerate to the center of the member. Moreover, even if a large amount/number of members for breast reconstruction are successfully placed, the individual members for breast reconstruction shift due to changes in the patient's position or external forces and tend to deform the implantation portion, making it difficult to regenerate a well-shaped breast.

The present invention aims to provide an adipose tissue regeneration substrate that has high handleability and that enables regeneration of a large volume of adipose tissue in a normal shape.

### Solution to Problem

The present invention relates to an adipose tissue regeneration substrate including: a plurality of granular bodies made of a bioabsorbable material, each granular body having an inner space and having on a surface a plurality of openings leading to the inner space; and a bag-shaped body made of a bioabsorbable material, the bag-shaped body having openings and wrapping the granular bodies.

The present invention is described in detail below.

After intensive studies, the present inventors have found out that packing a plurality of granular bodies made of a bioabsorbable material in a bag-shaped body having openings and made of a bioabsorbable material can provide an adipose tissue regeneration substrate that is easy to implant in large resection sites. Such a regeneration substrate is less likely to deform due to external forces, and thus can regenerate tissue with a normal shape. The inventors thus completed the present invention.

The adipose tissue regeneration substrate of the present invention includes a plurality of granular bodies made of a bioabsorbable material, each granular body having an inner space and having on a surface a plurality of openings leading to the inner space, and a bag-shaped body made of a bioabsorbable material, the bag-shaped body having openings and wrapping the granular bodies.

FIG. 1 is a schematic view of the adipose tissue regeneration substrate of the present invention. FIG. 2 is a schematic view of one of the granular bodies.

As shown in FIG. 1, the adipose tissue regeneration substrate of the present invention includes a plurality of granular bodies 1 enclosed in a bag-shaped body 3. Each granular body 1 has an inner space and has on a surface a plurality of openings leading to the inner space. Each granular body 1 has a closed shape as a whole. The bag-shaped body 3 has many openings and an inner space and has a closed shape to prevent its contents from moving out of the bag. Cells passing through the openings of the bag-shaped body 3 and the openings of the granular bodies 1 use wall surfaces inside the granular bodies as scaffolds to proliferate, thus regenerating adipose tissue. The granular bodies 1 and the bag-shaped body 3, each made of a bioabsorbable material, maintain the space for the adipose tissue to regenerate until the adipose tissue has regenerated. After the adipose tissue has regenerated, the granular bodies 1 and the bag-shaped body 3 are absorbed into the body and eventually disappear. With the granular bodies held together in the bag-shaped body, the adipose tissue regeneration substrate of the present invention is easy to implant in large spaces and thus has high handleability. Moreover, the granular bodies 1, wrapped in the bag-shaped body 3, do not scatter over a wide area, so that the adipose tissue regeneration substrate can be implanted in a shape close to the shape after regeneration. If receiving an external force after implantation, the granular bodies 1 remain inside the bag-shaped body 3, so that the shape at the time of implantation is less likely to be lost, enabling regeneration of adipose tissue with a normal shape. The adipose tissue regeneration substrate of the present invention may further include a sponge-like porous body 2 made of a bioabsorbable material inside each of the granular bodies 1. The sponge-like porous body 2 made of a bioabsorbable material in each of the granular bodies 1 provides more scaffolds for cells, promoting regeneration of adipose tissue while also increasing strength.

The sponge-like porous body encompasses not only a spongy body but also a body with a shape having many voids, such as non-woven fabric or cotton.

The bioabsorbable material constituting the granular bodies may be any bioabsorbable material confirmed to be safe as an implant. As regeneration of adipose tissue takes about six months to a year, the bioabsorbable material preferably has strength and a decomposition rate to maintain the space where the adipose tissue regeneration substrate is implanted for the period of about six months to a year. Examples of such a bioabsorbable material include: natural polymers such as collagen, gelatin, chitin, and chitosan; and synthetic polymers such as homopolymers of lactic acid, glycolic acid, ε-caprolactone, dioxanone, and trimethylene carbonate and copolymers of at least two materials selected from them. In particular, the bioabsorbable material is preferably polylactic acid or a copolymer of lactic acid and another bioabsorbable material because their strength and in-vivo decomposition rate are suitable for the adipose tissue regeneration substrate. Examples of the polylactic acid or the copolymer of lactic acid and another bioabsorbable material include polylactide disclosed in Patent Literature 1, copolymers of lactide and glycolic acid, and copolymers of lactide and ε-caprolactone.

When the bioabsorbable material of the granular bodies is polylactide, a copolymer of lactide and glycolic acid, or a copolymer of lactide and ε-caprolactone, the bioabsorbable material preferably has a weight average molecular weight of 4000 or greater and 300,000 or less. The weight average molecular weight within the above range allows the decomposition rate to be more suitable for regeneration of adipose tissue. The weight average molecular weight is more preferably 100,000 or greater, more preferably 200,000 or less.

The granular bodies may have any shape that can provide a scaffold for cell proliferation and maintain the space for the adipose tissue to regenerate. Examples include spherical, columnar, and irregular shapes. In particular, the granular bodies are preferably spherical, more preferably ellipsoidal so that they can be less likely to lose their shape due to external forces after implantation and can have appropriate gaps between the granular bodies to further promote regeneration of adipose tissue.

The inner space may have any size. The size is preferably 10 mm³ or greater and 100,000 mm³ or less. The inner space having a size within the range can secure the space for regenerating adipose tissue while allowing adipose tissue to more reliably regenerate to the center of the granular body. The size of the inner space is more preferably 25 mm³ or greater, still more preferably 50 mm³ or greater and is more preferably 50,000 mm³ or less, still more preferably 25,000 mm³ or less.

The openings of the granular bodies may have any shape and may be circular, grid-shaped, polygonal, or irregularly shaped.

Each granular body may have any number of openings not less than two. The openings of each granular body may have any size and may occupy any percentage of the surface area as long as cells can smoothly pass through the openings into the granular body. Preferably, openings having a maximum length of 0.1 mm or greater and 20 mm or less are distributed to occupy 50% or greater and 99% or less of the surface area of the granular body. The size of the openings and the percentage of the surface area occupied by the openings within the above ranges allow an increased balance between the strength of the granular body and the ease of entering of cells. Each opening has a maximum length that allows entering of adipose tissue but prevents entering of the surrounding, already existing tissues other than adipose. The maximum length is more preferably 15 mm or less, still more preferably 10 mm or less. The openings more preferably occupy 60% or greater, still more preferably 70% or greater of the surface area of the granular body to facilitate entering of tissue. The openings more preferably occupy 95% or less, still more preferably 90% or less of the surface area of the granular body to secure the shape of the granular body. The maximum length herein refers to the maximum distance measured between two points of an opening.

More specific embodiments of the granular bodies include mesh granular bodies and porous capsules. For mesh granular bodies, examples of the mesh constituting the granular bodies include nets formed of monofilaments or multifilaments, woven fabrics, and knitted fabrics. From the standpoint of properties such as elasticity, shape retainability, and ease of entering of adipose tissue, mesh ellipsoidal bodies are more preferred.

When the granular bodies are made of a mesh, each of the individual threads of the mesh constituting the granular bodies may have any thickness. From the standpoint of properties of the mesh such as elasticity, shape retainability, and ease of entering of cells, the thickness is preferably 0.05 mm or greater and 1 mm or less, more preferably 0.1 mm or greater and 0.4 mm or less. The mesh constituting the granular bodies preferably has an aperture size of 0.01 mm or greater and 6 mm or less, more preferably 0.02 mm or greater and 5 mm or less in length and width.

The granular bodies each may have any size. When the granular bodies are ellipsoidal, the major axis is preferably 8 mm or greater and 150 mm or less, and the minor axis is preferably 5 mm or greater and 100 mm or less. The size of each of the granular bodies within the range makes it easy to adjust the shape when regenerating a large volume of adipose tissue and also allows adipose tissue to more reliably regenerate to the center of the granular body. The major axis of each of the granular bodies is more preferably 10 mm or greater and 30 mm or less, still more preferably 15 mm or greater and 20 mm or less. The minor axis of each of the granular bodies is more preferably 5 mm or greater and 20 mm or less, still more preferably 7 mm or greater and 15 mm or less.

The adipose tissue regeneration substrate of the present invention may include any number of granular bodies not less than two. The number of granular bodies can be appropriately adjusted according to the size of the granular bodies and the size of the space in which the substrate is to be implanted. From the standpoint of handleability and further promoting regeneration of a large volume of adipose tissue, the adipose tissue regeneration substrate preferably includes 5 or more, more preferably 10 or more but preferably 100 or less, more preferably 50 or less granular bodies.

The bioabsorbable material constituting the sponge-like porous body may be any bioabsorbable material. Examples thereof include: synthetic polymers such as polyglycolide, polylactide, poly-ε-caprolactone, lactide-glycolic acid copolymers, glycolide-ε-caprolactone copolymers, lactide-ε-caprolactone copolymers, polycitric acid, polymalic acid, poly-α-cyanoacrylate, poly-β-hydroxy acid, polytrimethylene oxalate, polytetramethylene oxalate, polyorthoester, polyorthocarbonate, polyethylene carbonate, poly-γ-benzyl-L-glutamate, poly-γ-methyl-L-glutamate, poly-L-alanine, and polyglycol sebacic acid; and natural polymers such as polysaccharides (e.g., starch, alginic acid, hyaluronic acid, chitin, pectic acid, and derivatives thereof) and proteins (e.g., gelatin, collagen, albumin, and fibrin). The bioabsorbable material preferably contains collagen, which is highly compatible with the living body.

When the sponge-like porous body contains collagen, the sponge-like porous body preferably contains collagen in an amount of 50% by weight or more. The amount of collagen in the sponge-like porous body is more preferably 60% by weight or more, still more preferably 70% by weight or more, further preferably 80% by weight or more, particularly preferably 90% by weight or more, very preferably 95% by weight or more, most preferably 100% by weight.

The collagen may be any collagen derived from the skin, tendons, or other sites of cows, pigs, and the like. In particular, to eliminate antigenicity and increase safety, atelocollagen is preferred. Atelocollagen can be obtained by treating collagen with an enzyme such as protease or pepsin to remove telopeptide as much as possible.

Examples of commercially available sponge-like porous bodies containing collagen include Pelnac (produced by Smith & Nephew Wound Management KK.) and Terudermis (produced by Terumo Corporation).

The present invention also encompasses a granular body used for the adipose tissue regeneration substrate of the present invention described above, the granular body being made of a bioabsorbable material, having an inner space, and having on a surface a plurality of openings leading to the inner space.

The bag-shaped body may have any shape, such as a rectangular bag shape or a circular bag shape, according to the ease of molding at implantation sites. Specific embodiments include bag-shaped nets knitted using filaments and porous bag-shaped bodies.

The bioabsorbable material constituting the bag-shaped body may be any bioabsorbable material. It may be the same bioabsorbable material as that constituting the sponge-like porous body because the bag-shaped body does not need to maintain its strength for as long a time as the granular bodies. Still, the bag-shaped body needs strength to hold the granular bodies and maintain the shape of the entire substrate. Since the bag-shaped body is implanted in the living body, the bioabsorbable material preferably causes as little inflammation or foreign body reaction as possible. Examples of such a bioabsorbable material include material usable as sutures. The bioabsorbable material used is preferably polyglycolide, polylactic acid, polycaprolactone, polydioxane, trimethylene carbonate, or a copolymer containing any of these, more preferably polyglycolide, a copolymer of polyglycolide and another bioabsorbable material, or a copolymer of lactic acid and another bioabsorbable material.

When the bag-shaped body is a net, the filaments constituting the bag-shaped body may have any thickness. From the standpoint of the balance between flexibility and strength, the thickness is preferably 0.01 mm or greater, more preferably 0.1 mm or greater and is preferably 2 mm or less, more preferably 0.5 mm or less.

The openings of the bag-shaped body may occupy any percentage of the surface area as long as cells can pass through the openings to reach the granular bodies. The openings preferably occupy 50% or greater and 99% or less of the surface area of the bag-shaped body. The percentage occupied by the openings within the range allows a more increased balance between the strength and cell penetrability of the bag-shaped body. The openings of the bag-shaped body more preferably occupy 60% or greater, still more preferably 70% or greater and more preferably 95% or less, still more preferably 90% or less of the surface area of the bag-shaped body.

The openings of the bag may have any size that do not inhibit the cells entering the granular bodies and prevents the granular bodies from falling or protruding out of the bag-shaped body. The openings of the bag-shaped body preferably have a maximum length that is at least 1/50 times, more preferably at least 1/20 times and preferably at most ⅓ times, more preferably at most 1/10 times the minor axis of the granular bodies. The size of the openings within the range can increase the shapeability and handleability of the entire adipose tissue regeneration substrate to be obtained.

When the bag-shaped body is a net, the bag-shaped body specifically preferably has an aperture size of, for example, 0.02 mm or greater and 0.5 mm or less, more preferably 0.05 mm or greater and 0.1 mm or less both in length and width.

The size of the bag-shaped body can be appropriately adjusted according to the volume of the implantation site and the number of granular bodies. To increase the moldability of the adipose tissue regeneration substrate while preventing the implanted granular bodies from collapsing, the inner space of the bag-shaped body is preferably at least 1.2 times, more preferably at least 1.5 times and preferably at most 3 times, more preferably at most 2 times the total volume of the granular bodies. The total volume of the granular bodies includes the volume of the inner space of each granular body.

The method for producing the adipose tissue regeneration substrate of the present invention is not limited. For example, the adipose tissue regeneration substrate can be produced by: producing a plurality of granular bodies by wrapping the sponge-like porous bodies with meshes made of a bioabsorbable material and closing the ends of each mesh; wrapping the obtained granular bodies in a bag-shaped body made of a bioabsorbable material; and closing the end of the bag-shaped body. Alternatively, after the granular bodies are produced, the sponge-like porous bodies may be inserted from the openings of the granular bodies. The method for closing the ends of the mesh or the bag-shaped body is not limited. For example, filaments may be tied together, or thermal pressure bonding may be performed.

The adipose tissue regeneration substrate of the present invention is implanted in adipose tissue and used to regenerate adipose tissue. The present invention enables regeneration of living adipose tissue from autologous cells without implantation of tissue from other sites. Examples of adipose tissue for which the present invention can be used include that of the breasts, the buttocks, and the abdomen. The present invention can regenerate a large volume of adipose tissue in a normal shape. The present invention thus has a great effect particularly in applications where a breast is regenerated by implanting the adipose tissue substrate in a defect caused by partial mastectomy.

### Advantageous Effects of Invention

The present invention can provide an adipose tissue regeneration substrate that has high handleability and that enables regeneration of a large volume of adipose tissue in a normal shape.

### BRIEF DESCRIPTION OF DRAWINGS

FIG. 1 is a schematic view of the adipose tissue regeneration substrate of the present invention.

FIG. 2 is a schematic view of a granular body.

FIG. 3 is a graph showing measurement results of moldability for a hole-shaped defect.

FIG. 4 is a graph showing measurement results of moldability for a horizontal defect.

FIG. 5 shows magnetic resonance images (MRIs) of adipose tissue regeneration substrates obtained in Example 1, taken zero (immediately after implantation), one, three, six, and nine months after implantation in fascial defects of a pig.

FIG. 6 is an image of a hematoxylin-eosin (HE) stained implantation portion, taken six months after the adipose tissue regeneration substrates obtained in Example 1 were implanted in the fascial defects of the pig.

FIG. 7 is an image of an oil red O stained implantation portion, taken six months after the adipose tissue regeneration substrates obtained in Example 1 were implanted in the fascial defects of the pig.

FIG. 8 is an image of an Azan stained implantation portion, taken six months after the adipose tissue regeneration substrates obtained in Example 1 were implanted in the fascial defects of the pig.

FIG. 9 is an image of anti-CD31 antibody immunostained implantation portion, taken six months after the adipose tissue regeneration substrates obtained in Example 1 were implanted in the fascial defects of the pig.

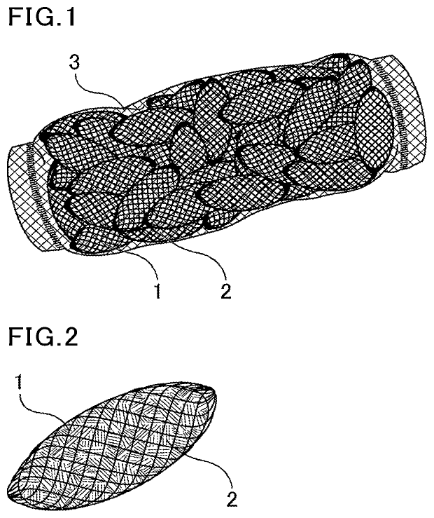

### DESCRIPTION OF EMBODIMENTS

In the following, embodiments of the present invention are described in detail with reference to examples. The present invention is not limited to these examples.

### Example 1

A collagen sponge (produced by Pelnac, Smith & Nephew Wound Management KK.) was wrapped in a mesh (filament thickness: 0.2 mm to 0.25 mm, mesh opening: 1×1 mm to 2×2 mm) made of polylactic acid (weight average molecular weight: 220,000), and the ends of the mesh were closed by thermal pressure bonding to give an ellipsoidal granular body having the collagen sponge inside and having a major axis of 18 mm and a minor axis of 7.5 mm. Thirty granular bodies were prepared in the same manner. The granular bodies were wrapped in a 110 mm×35 mm bag-shaped body having an envelope shape and made of polyglycolide multifilaments (filament structure: 0.015 mm×12, aperture size: 0.05 mm×0.05 mm). The end of the bag-shaped body was closed by thermal welding, whereby an adipose tissue regeneration substrate was obtained.

### Example 2

An adipose tissue regeneration substrate was obtained as in Example 1 except that no collagen sponge was used.

### Comparative Example 1

The 30 granular bodies of Example 1 were directly used as adipose tissue regeneration substrates.

### <Evaluation>

The adipose tissue regeneration substrates obtained in the examples and the comparative example were evaluated as follows.

### (Tissue Regeneration Evaluation 1)

A subcutaneous incision was made in the back of a miniature pig (about 20 kg). The adipose tissue regeneration substrate obtained in Example 1 or 2 was implanted to the left of the midline. After four months, the portion in which the adipose tissue regeneration substrate was implanted was removed and examined for tissue regeneration. About 4 cm of tissue was regenerated.

### (Tissue Regeneration Evaluation 2)

A miniature pig (about 25 kg), a large animal, was provided as an experimental animal. A median incision was made in the abdominal skin. Subsequently, the adipose and mammary tissue on the left and right sides of the abdomen were removed, and defects were made on the fascia under the mammary gland. In each of the fascial defects was implanted the adipose tissue regeneration substrate obtained in Example 1, and the skin was sutured.

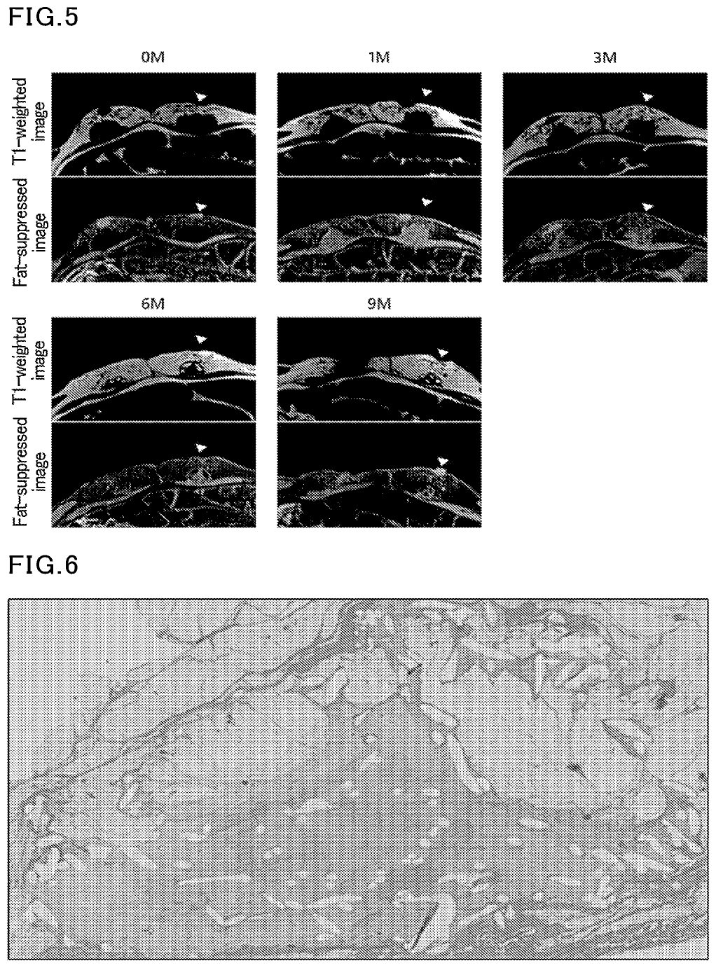

Magnetic resonance images (MRIs) of the abdomen were taken zero (immediately after implantation), one, three, six, and nine months after surgery. FIG. 5 shows the magnetic resonance images (MRIs).

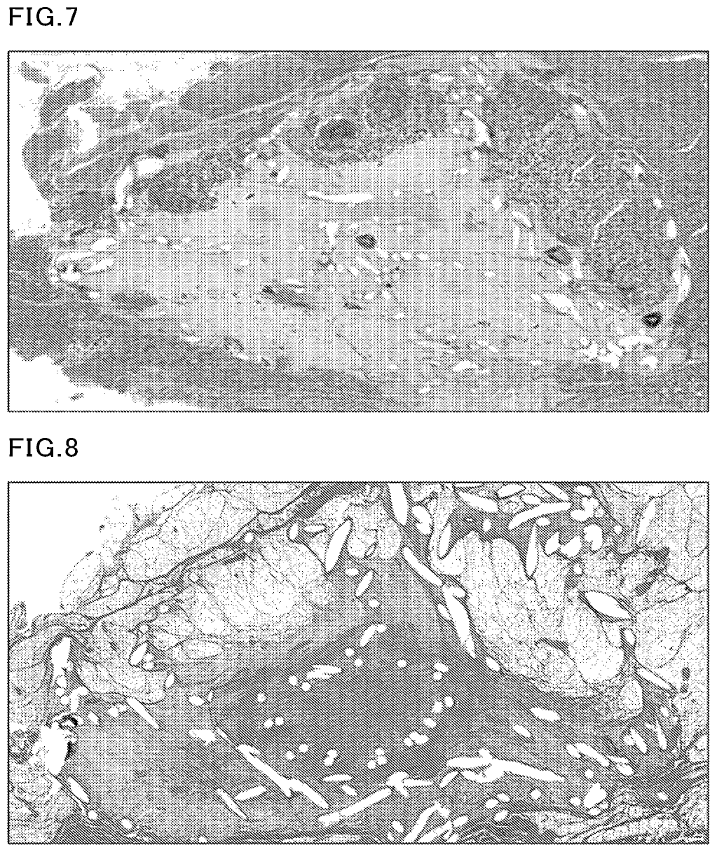

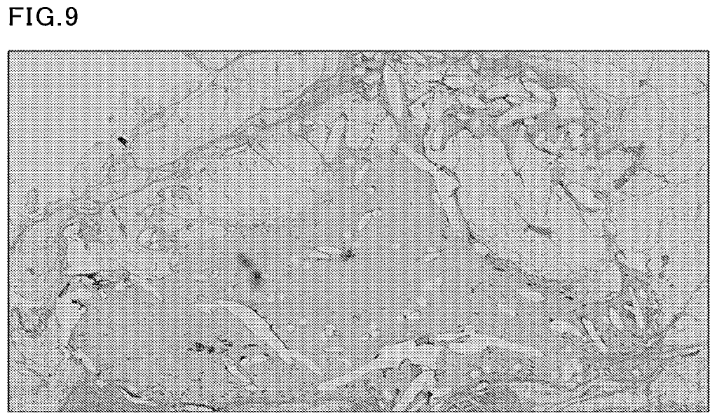

Six months after surgery, adipose tissue on the muscular layer on the right side of the abdomen was taken out, and the implantation portion was removed. The obtained specimen was sectioned and subjected to hematoxylin-eosin (HE) staining, oil red O staining, Azan staining, and anti-CD31 antibody immunostaining. The micrographs of the HE stained, oil red O stained, Asan stained, and anti-CD31 antibody immunostained sections are respectively shown in FIG. 6, FIG. 7, FIG. 8, and FIG. 9.

FIG. 5 demonstrates that six months after surgery, the portions in which the adipose tissue regeneration substrates were implanted showed regeneration of adipose tissue (white portions in the MRI (T1-weighted image in FIG. 5) from the peripheral portions adjoining adipose tissue or mammary tissue. Nine months after surgery, regeneration of adipose tissue was observed in a larger area from the peripheral portion of the adipose tissue regeneration substrate.

Furthermore, FIG. 6, FIG. 7, and FIG. 8 show that six months after surgery, adipose tissue and collagen tissue were formed inside the adipose tissue regeneration substrate. FIG. 9 shows that blood vessels were formed in this adipose tissue and collagen tissue.

### (Evaluation of Moldability)

### (1) Moldability for Hole-Shaped Defect

Unskinned chicken breast (358 g) was provided as a substitute for the skin and adipose tissue. The skin of the chicken breast was partially peeled to expose the meat. Subsequently, a cross-shaped incision was made in the exposed meat, and the center of the incision was hollowed out to form a hole-shaped defect. The skin was put back to the original position before the longitudinal and transverse lengths of the incision and the height of the defect were measured. The adipose tissue regeneration substrate obtained in Example 1 was then implanted in the defect. The skin was put back to the original position, and then the longitudinal and transverse lengths of the incision and the height of the defect were measured.

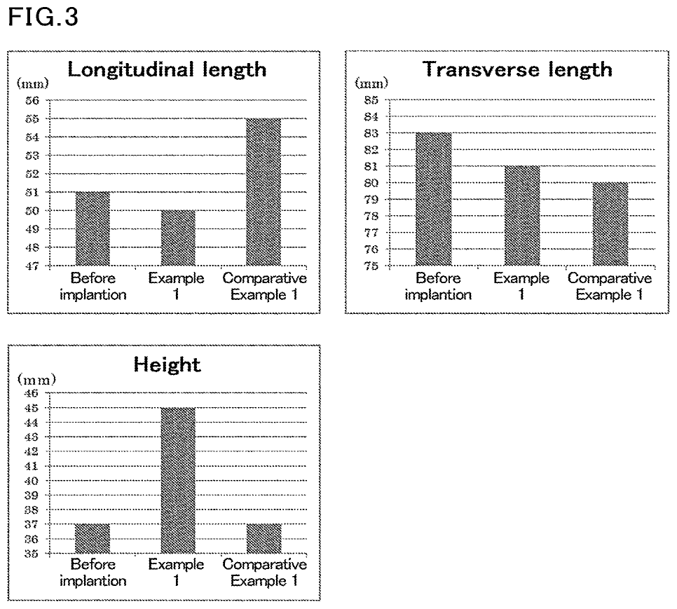

Subsequently, the 30 adipose tissue regeneration substrates of Comparative Example 1 were used, and the longitudinal and transverse lengths of the incision and the height of the defect were measured in the same manner. FIG. 3 shows the measurement results. The measurement results show that the adipose tissue regeneration substrates of Comparative Example 1 are difficult to mold in the height direction because they enter the incision, whereas the adipose regeneration substrate of Example 1 is easy to mold into a tall shape because the adipose tissue regeneration substrate does not much spread in a longitudinal or transverse direction, and the granular bodies are close together as if they form a mountain in the height direction. This shows that the adipose tissue regeneration substrate of Example 1 has excellent moldability for adipose tissue of the breasts and the buttocks, for example.

### (2) Moldability for Horizontal Defect

Unskinned chicken breast (379 g) was provided as a substitute for the skin and adipose tissue. The skin was partially peeled to expose the meat. One incision was made along the muscle fiber direction of the exposed meat. The transverse length of the incision, the longitudinal length of the incision when the incision was opened, and the height of the incision were measured. The adipose tissue regeneration substrate obtained in Example 1 was then implanted in the incision, and the longitudinal and transverse lengths and the height of the incision were measured. Subsequently, the 30 adipose tissue regeneration substrates of Comparative Example 1 were used, and the longitudinal and transverse lengths and the height of the incision were measured in the same manner.

At this time, observation of the states of the implanted adipose tissue regeneration substrates of Example 1 and Comparative Example 1 showed that the adipose tissue regeneration substrate of Example 1 did not protrude from the incision, whereas multiple adipose tissue regeneration substrates of Comparative Example 1 protruded or fell off from the incision. Here, the measurement in Comparative Example 1 was performed after the protruding or fallen adipose tissue regeneration substrates were pushed back into the incision.

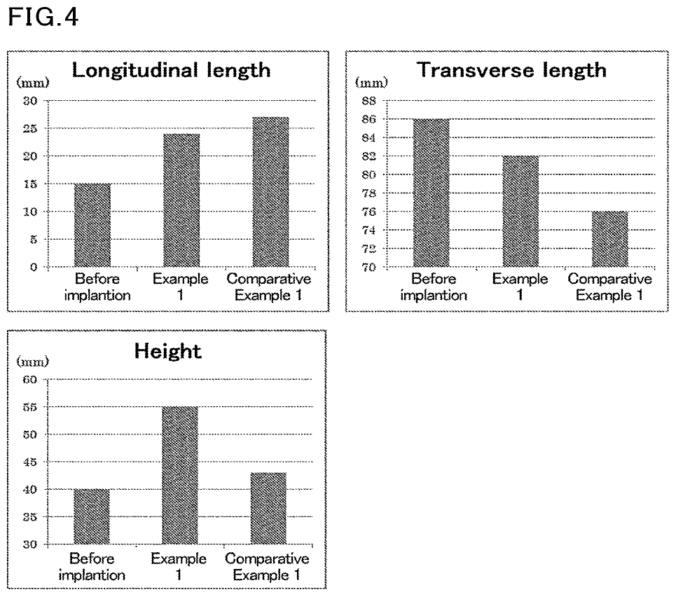

FIG. 4 shows the measurement results. The measurement results show that the adipose tissue regeneration substrates of Comparative Example 1 are difficult to mold in the height direction because they spread in the longitudinal direction of the incision, whereas the adipose tissue regeneration substrate of Example 1 is easy to mold into a tall shape because the adipose tissue regeneration substrate does not much spread in the longitudinal or horizontal direction, and the granular bodies are close together as if they form a mountain in the height direction. This shows that the adipose tissue regeneration substrate of Example 1 has excellent moldability for adipose tissue of the breasts and the buttocks, for example.

### INDUSTRIAL APPLICABILITY

The present invention can provide an adipose tissue regeneration substrate that has high handleability and that enables regeneration of a large volume of adipose tissue in a normal shape.

### REFERENCE SIGNS LIST

1 Granular body
2 Sponge-like porous body
3 Bag-shaped body

1. An adipose tissue regeneration substrate comprising:
a plurality of granular bodies made of a bioabsorbable material, each granular body having an inner space and having on a surface a plurality of openings leading to the inner space; and
a bag-shaped body made of a bioabsorbable material, the bag-shaped body having openings and wrapping the granular bodies.

2. The adipose tissue regeneration substrate according to claim 1,
wherein the granular bodies are ellipsoidal bodies containing polylactic acid or a copolymer of lactic acid and another bioabsorbable material.

3. The adipose tissue regeneration substrate according to claim 1, comprising a sponge-like porous body made of a bioabsorbable material inside each of the granular bodies.

4. The adipose tissue regeneration substrate according to claim 1,
wherein the bioabsorbable material constituting the bag-shaped body is polyglycolide, a copolymer of polyglycolide and another bioabsorbable material, or a copolymer of lactic acid and another bioabsorbable material.

5. The adipose tissue regeneration substrate according to claim 1, used for implantation in a defect caused by partial mastectomy.

6. A granular body used for the adipose tissue regeneration substrate according to claim 1, the granular body being made of a bioabsorbable material, having an inner space, and having on a surface a plurality of openings leading to the inner space.

7. The granular body according to claim 6, which is an ellipsoidal body containing polylactic acid or a copolymer of lactic acid and another bioabsorbable material.

8. The granular body according to claim 6, comprising a sponge-like porous body made of a bioabsorbable material inside the granular body.
